(19) United States
(12) Patent Application Publication
(10) Pub. No.: US 2005/0176062 A1
(43) Pub. Date: Aug. 11, 2005

(54) Method of identifying ligands for nuclear receptors

(76) Inventors: Reiner Aichholz, Basel (CH); Francis Bitsch, Dietwiller (FR); Sabine Geisse, Weil (DE); Jean-Marc Schlaeppi, Allschwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/512,891

(22) PCT Filed: Apr. 28, 2003
(86) PCT No.: PCT/EP03/04429

Related U.S. Application Data
(60) Provisional application No. 60/376,434, filed on Apr. 29, 2002.

(51) Int. Cl. G01N 33/53; G01N 33/00; C12N 15/85

(52) U.S. Cl. 435/7.1; 435/455; 436/86

(57) ABSTRACT

The present invention provides a method of identifying a ligand to a nuclear receptor protein comprising wherein said nuclear receptor protein is first expressed in an eukaryotic expression system in the presence of at least one candidate ligand. The nuclear receptor protein is then purified, isolated and mass spectrometry is used to determine the presence of a bound ligand. The ligand is finally isolated and the measured spectra is compared with the spectra of known ligands in compound libraries to identify the ligand.

### FIELD OF THE INVENTION

The invention relates to a method of identifying ligands to nuclear receptors, including orphan nuclear receptors.

### BACKGROUND OF THE INVENTION

Nuclear receptors represent a superfamily of proteins that allow specific binding of physiologically relevant small molecule ligands, such as hormones or vitamins. Nuclear receptors act as ligand-inducible transcription factors which directly interact as monomers, homodimers or heterodimers with DNA response elements of target genes as well as through signaling pathways. Members of the nuclear receptor superfamily include receptors such as those for glucocorticoids (GRs), androgens (ARs), mineralocorticoids (MRs), progestins (PRs), estrogens (ERs), thyroid hormones (TRs), vitamin D (VDRs), retinoids (RARs and RXRs), peroxisomes (XPARs and PPARs) and icosanoids (IRs).

Unlike integral membrane receptors and membrane associated receptors, nuclear receptors reside in either the cytoplasm or nucleus of eukaryotic cells. Thus, nuclear receptors comprise a class of intracellular, soluble ligand-regulated transcription factors which are found only in eukaryotic cells. Members of this family display an overall structural motif of three modular domains: (i) a variable amino-terminal domain, (ii) a highly conserved DNA-binding domain (DBD) and (iii) a less conserved carboxyl-terminal ligand binding domain (LBD). The modularity of this superfamily permits different domains and subdomains of each protein to separately accomplish different functions, although the domains can influence each other. The separate function of a domain is usually preserved when a particular domain (e.g. ligand binding domain) is isolated from the remainder of the protein. The so-called “orphan nuclear receptors” are also part of the nuclear receptor superfamily as they are structurally homologous but have not yet been identified to be associated with specific ligands.

### SUMMARY OF THE INVENTION

The present invention provides a method-of identifying a ligand to a nuclear receptor protein comprising:

(i) expressing a nuclear receptor protein in an eukaryotic expression system in the presence of at least one candidate ligand

(ii) purifying and isolating the nuclear receptor protein

(iii) measuring the spectra and molecular weight of the protein and protein-ligand complex by mass spectrometry to determine the presence of the ligand

(iv) isolating the ligand and comparing the measured mass spectra of the ligand with the mass spectra of compounds in known compound libraries to identify the ligand.

In one embodiment of the invention, a method is provided wherein said nuclear receptor protein comprises the ligand binding domain of a nuclear receptor protein.

In a preferred embodiment of the invention, the expression of the nuclear receptor protein occurs in a baculovirus expression system using insect cells.

In another embodiment of the invention, the expression of the nuclear receptor may occur in the presence of at least one coregulator. In a further embodiment of the invention, purification and isolation of the nuclear receptor protein may also occur in the presence of at least one coregulator.

According to the invention, the mass spectrometry method for determining the presence of the ligand preferably comprises continuous or pulsed electrospray ionization, or matrix assisted laser desorption ionization (MALDI).

According to the invention, the mass spectrometry method for identifying the ligand preferably comprises continuous or pulsed electrospray ionization (ESI), atmospheric pressure chemical ionization (APCI), matrix assisted laser desorption ionization (MALDI), electron impact ionization (EI) or chemical ionization (Cl) mass spectrometry.

According to the invention, the ligand is identified from a library comprising small molecule organic compounds.

Further according to the invention, said method of identifying ligands to nuclear receptor proteins may be used in an automated high throughput screen.

### DESCRIPTION OF THE INVENTION

The present invention relates to nuclear receptors including the orphan nuclear receptors since proteins of the nuclear receptor superfamily display an overall structural similarity, particularly in three modular domains:(i) a variable amino-terminal domain, (ii) a highly conserved DNA-binding domain (DBD); and (iii) a less conserved carboxyl-terminal ligand binding domain (LBD). The modularity of this superfamily permits different domains of each protein to separately accomplish different functions, although the domains can influence each other. The separate function of a domain is usually preserved when a particular domain is isolated from the remainder of the protein. Isolation of a protein or a domain from a protein can be accomplished by conventional protein chemistry techniques. Using conventional molecular biology techniques a domain or subdomain can be separately expressed with its original function intact. Chimerics of two or more different nuclear receptors can also be constructed, wherein the chimerics retain the properties of the individual functional domains of the respective nuclear receptors from which the chimerics were generated. Therefore, the present invention relates not only to nuclear receptor proteins but also to isolated domains of nuclear receptors proteins.

The present invention preferably relates to a ligand binding domain (LBD) of a nuclear receptor. The LBD is a conserved domain in nuclear receptors. Whereas integrity of several different LBD sub-domains is important for ligand binding, truncated molecules containing only the LBD retain normal ligand-binding activity. This domain also participates in other functions, including dimerization, nuclear translocation and transcriptional activation. Importantly, this domain binds the ligand and undergoes ligand-induced conformatlonal changes.

According to the invention, the method of identifying a ligand to a nuclear receptor protein involves expression of a nuclear receptor protein or nuclear receptor ligand binding domain in eukaryotic cells. Appropriate host cells are eukaryotic cells capable of expressing the cloned sequence. Preferably, the eukaryotic cells are cells of higher eukaryotes. Suitable eukaryotic cells include non-human mammalian tissue culture cells and human tissue culture cells. Preferred host cells include insect cells,

such as Sf21, Sf9 which is a clonal derivative of Sf21, and Hi five; Chinese hamster ovary cells (CHO), human embryonic kidney cells (HEK 293), EBNA 1 transformed HEK 293 cells (HEK.EBNA cells), murine 3T3 fibroblasts, African Green monkey kidney cells (COS cells), baby hamster kidney cells (BHK), mouse myeloma cells (Sp2/0 and NS0), and human cervix carcinoma cell line (HeLa).

As host cells for recombinant protein expression, eukaryotes possess the advantage of generating correctly folded proteins carrying secondary modifications required and potentially essential for biological function. With respect to the present invention, eukaryote host cells further can provide a natural source of the ligand, thus stabilizing the nuclear receptor to be expressed.

According to the invention, most preferred are insect cells as host cells. In a preferred embodiment of the invention, a nuclear receptor protein or nuclear receptor protein domain is expressed using a baculovirus expression system using insect cells (for techniques of baculovirus expression see Luckow et al., Bio/Technology, 1988, 6, 47, Baculovirus Expression Vectors: A Laboratory Manual, O'Rielly et al. (Eds.), W. H. Freeman and Company, New York, 1992, and U.S. Pat. No. 4,879,236).

A baculovirus expression system using insect cells has advantages compared to other expression systems known in the art. The time from cloning of the gene to production of the protein is short compared to the time needed to establish a stably transformed animal cell line, especially when associated with gene amplification procedures. In addition, cell death inevitably follows virus infection of insect cells. This can be an advantage over other expression systems because it may permit better expression of cytotoxic, regulatory or essential cellular genes such as those of nuclear receptor proteins.

According to the invention, several insect cell-lines are suitable for infection with a recombinant baculovirus. For example, the cell line Sf-21 derived from ovarial tissue of the fall armyworm (Spodoptera frugiperda), Sf-9, a clonal derivative of Sf-21, available from the American Type Culture Collection (CRL 1711), the Hi-Five cell-line and the Tn-368 and Tn-368A cell-lines obtained from the cabbage looper (Trichoplusia ni). The most widely used media in which insect cells grow include TNM-FH and IPL-41. These media are usually supplemented with more or less defined components, such as mammalian sera, in particular foetal calf serum. Serum replacements have also been applied to insect-cell culture, and serum-free media, such as Ex-Cell 401, Ex-Cell 405 and SF 900 II are commercially available and currently widely applied to facilitate protein purification.

According to the invention, the nuclear receptor protein also may be coexpressed in the presence of at least one coregulator. Further, according to the invention, the nuclear receptor protein may be purified or isolated in the presence of at least one coregulator. Coregulators are molecules which mediate the binding of small molecular ligands to a nuclear receptors. As a result of a specific binding of the small molecule ligand to a nuclear receptor, the nuclear receptor changes the ability of a cell to transcribe DNA. Coregulators are typically divided into classes of coactivators and corepressors depending on the particular nuclear receptor protein. Coactivators are molecules which promote the activation of transcription factors while corepressors inhibit the formation of transcriptionally active complexes. A subset of nuclear receptors may bind corepressor factors and actively repress target gene expression in the absence of a ligand (Aranda et al., Physiological Reviews, Vol. 81, No. 3, July 2001).

According to the invention, co-activators such as those reviewed by Shibata, H., et al. (Recent Progress in Hormone Res. 52:141-164,1997) including steroid receptor co-activator-one (SRC-1), the SRC-1 related proteins, TIF-2 and GRIP-1, and other co-activators such as ARA-70, Trip 1, RIP-140, and TIF-1 may be used. Combinations of coactivators such as CBP and SRC-1 which are known to interact and synergistically to enhance transcriptional activation or a ternary complex of CBP, SRC-1, and liganded receptors may also be used.

Further, co-repressors such as SMRT and N-CoR may be used. Upon binding of an agonist, the receptor changes its conformation in the ligand-binding domain that enables recruitment of co-activators, which allows the receptor to interact with the basal transcriptional machinery more efficiently and to activate transcription. In contrast, binding of antagonists induces a different conformational change in the receptor. Although some antagonist-bound receptors can dimerize and bind to their cognate DNA elements, they fail to dislodge the associated co-repressors, which results in a nonproductive interaction with the basal transcriptional machinery.

In the case of mixed agonist/antagonists activation of gene transcription may depend on the relative ratio of co-activators and co-repressors in the cell or cell-specific factors that determine the relative agonistic or antagonistic potential of different compounds. These co-activators and co-repressors appear to act as an accelerator and/or a brake that modulates transcriptional regulation of hormone-responsive target gene expression.

According to the invention, isolation of the nuclear receptor protein can be made by a liquid chromatography system, which is optionally preceded by a cell lysis step (for intracellular expressed proteins). Preferred liquid chromatography fractionation step comprises affinity based chromatography (for tagged proteins) or immuno-affinity chromatography, size exclusion chromatography or ion exchange chromatography, all of which techniques are known to the person skilled in the art.

According to the invention, isolation of the nuclear receptor ligand can be made by a liquid or a gas chromatography separation, which is optionally preceded by an extraction step which removes the high molecular weight background. Preferred liquid chromatography separation steps comprise HPLC, reversed phase HPLC, ion-exchange chromatography (IE), capillary electrophoresis (CE), capillary electrochromatography (CEC), isoelectric focusing (IEF) or micellar electrokinetic chromatography (MEKC). Preferred gas chromatography separation steps comprises packed column gas chromatography, capillary gas chromatography or high temperature capillary gas chromatography. Preferred extraction steps comprise phase partitioning techniques such as solid phase extraction (SPE) and liquid phase extraction (LPE), all of which techniques are known to the person skilled in the art.

According to the invention, mass spectrometry formats for use in measuring the molecular weight and spectra of protein, ligand-protein complex and ligand include ionization techniques such as matrix assisted laser desorption (MALDI), continuous or pulsed electrospray (ESI) and related methods such as ionspray or thermospray, massive cluster impact (MCI), electron impact (EI) and chemical ionization (CI). Such ion sources can be matched with detection formats, including linear or non-linear reflectron time-of-flight (TOF), single or multiple quadrupole, single or multiple magnetic sector, Fourier transform ion cyclotron resonance (FTICR), ion trap, and combinations thereof such as ion-trap/time-of-flight. For ionization, numerous matrix/wavelength combinations (MALDI) or solvent combinations (ESI) can be employed. (Valaskovic, et al., Science 273:1199-1202 (1996); (Li et al., J. Am. Chem. Soc. 118:1662-1663(1996)).

Electrospray mass spectrometry has been described by Fenn et al. (J. Phys. Chem. 88:4451-59 (1984); PCT Application No. WO 90/14148) and current applications are summarized in review articles (Smith et al., Anal. Chem. 62:882-89 (1990); Ardrey, Electrospray Mass Spectrometry, Spectroscopy Europe 4:10-18 (1992)). MALDI-TOF mass spectrometry has been described by Hillenkamp et al. (“Matrix Assisted UV-Laser Desorption/Ionization: A New Approach to Mass Spectrometry of Large Biomolecules, Biological Mass Spectrometry” (Burlingame and McCloskey, eds., Elsevier Science Publ. 1990), pp. 49-60). ESI has been shown to enable the determination of the molecular weight of protein-ligand complexes (Veenstra et al., Biophys. Chem. 79:63-79 (1999). With ESI, the determination of molecular weights in femtomole amounts of sample is very accurate due to the presence of multiply-charged ion peaks, all of which can be used for mass calculation.

According to the invention, identification and/or structure determination of the ligand can be made by comparison of chromatography retention time or mass spectrum (MS or tandem mass spectrometry, eg. MS/MS) or both, with a known compound or by searching the mass spectra against a library of mass spectra such as the NIST MS database. Structural identification of the ligand may also be performed using spectroscopical techniques such as Nuclear Magnetic Resonance (NMR) and related techniques such as infrared spectroscopy (IR) as well as x-ray crystallography.

The method of the present invention can be used in an automated system of high-throughput screening of ligands to nuclear receptor proteins.

### DEFINITIONS ACCORDING TO THE INVENTION

The term “ligand” according to the invention, refers to a molecule or group of molecules that bind to one or more specific sites of a nuclear receptor. Representative ligands include, by way of illustration, carbohydrates, monosaccharides, oligosaccharides, polysaccharides, amino acids, peptides, oligopeptides, polypeptides, proteins, nucleosides, nucleotides, oligonucieotides, polynucleotides, including DNA and DNA fragments, RNA and RNA fragments and the like, lipids, retinoids, steroids, glycopeptides, glycoproteins, proteoglycans and the like, and synthetic analogues or derivatives thereof, including peptidomimetics, small molecule organic compounds and the like, and mixtures thereof.

The term “candidate ligand” according to the invention is a ligand whose affinity or specificity for a target nuclear receptor has not yet been determined. Any type of molecule that is capable of binding to a target nuclear receptor may be considered to be a candidate ligand.

The term “protein” according to the invention, may be used interchangeably herein when referring to a “polypeptide” or “peptide”. The term “protein,” as used herein, means at least two amino acids, or amino acid derivatives, including mass modified amino acids, that are linked by a peptide bond, which can be a modified peptide bond. A polypeptide can be translated from a nucleotide sequence that is at least a portion of a coding sequence, or from a nucleotide sequence that is not naturally translated due, for example, to its being in a reading frame other than the coding frame or to its being an intron sequence, a 3′ or 5′ untranslated sequence, or a regulatory sequence such as a promoter. A polypeptide also can be chemically synthesized and can be modified by chemical or enzymatic methods following translation or chemical synthesis. The terms “protein,” “polypeptide” and “peptide” are used interchangeably herein when referring to a translated nucleic acid, for example, a gene product.

The term “isolated” as used herein with respect to a nucleic acid, including DNA and RNA, refers to nucleic acid molecules that are substantially separated from other macromolecules normally associated with the nucleic acid in its natural state. An isolated nuclear receptor protein or domain of a protein according to the invention, refers to a protein or domain substantially separated from the cellular material normally associated with it in a cell. An isolated part of a nuclear receptor protein can be a fragment thereof that does not occur in nature. The term “isolated” also is used herein to refer to a ligand isolated from a protein-ligand complex.

The term “small molecule organic compound” according to the invention refers to organic compounds generally having a molecular weight less than about 1000, preferably less than about 500.

The “compound libraries” according to the invention typically contain a plurality of members or ligands. Further according to the invention, a compound library may also be a library of mass spectra of small molecular organic compounds against which the spectra of a ligand may be compared in order to identify the ligand.

According to the invention, compound libraries may contain racemic mixtures to determine, for example, if only one isomer (e.g. an enantiomer or diastereomer) is binding to the target receptor, or if the isomers have different affinities for the target receptor. In this regard, if the isomers have different affinities for the target receptor, a different break through time is to be observed for each isomer.

### EXAMPLES

### Example 1

### Cloning, Expression, Purification and Binding Assay of a Nuclear Receptor Ligand Binding Domain (His)6 RORa-LBD269-556 Expressed in Bacilovirus System

Sf9 and Sf21 cells are taken from Spodoptera frugiperda. A biotinylated 24-mer peptide (Biotinyl-GSTHGTSLKEKHKILHRLLQDSSS-NH2) corresponding to residues 676-699 of the mouse coactivator GRIP1 is synthesized by Mimotopes (Pty) Ltd (#814201). A shorter 20-mer peptide GTSLKEKHKILHRLLQDSSS is synthesized by Neosystems (#SP000267). The purity of both peptides are >95%. Biotinyl-DEVD-1-AL is from Sigma (#B7795). Streptavidin coated SA-chips, and HBS buffer (Biacore). Ni-NTA Superflow resin from Qiagen. Other chromatographic materials are from Amersham/Pharmacia Biotech.

Cloning

The nuclear receptor RORa ligand binding domain is cloned in the Baculovirus vector pBacPAK8-His1. The pBacPAK8 expression vector is from Clontech (Palo Alto, Calif., USA). The pCMX RORa1 was from (Serono Pharmaceuticals, Geneva, Switzerland). The RORa ligand-binding domain (LBD) is obtained by excising an EcoRV/BamH1 fragment corresponding to the RORa sequence 269-556. This fragment is inserted into the HincI/BamH1 sites of the vector pBacPAK8-His1 using the rapid DNA ligation kit (Boehringer Mannheim, Mannheim, Germany) and recombinant colonies containing pBacPAK8-RORa269-556 are identified. The construct is verified by sequencing.

Baculovirus Expression in Insect Cells

The recombinant RORa-LBD carrying transfer vector is co-transfected with linearised AcNPV viral DNA (BacPAK 6, Clontech) into Sf21 insect cells. After an incubation period of six days, the supematant containing recombinant virus is harvested and subjected to plaque-assay purification. Eight well-defined plaques are isolated; subsequently the virus is amplified on small scale, the infected cells are harvested and analysed for expression of RORa-LBD by Western Blot detection of the 6xHis-tag. All eight plaque isolates scoring positive for expression of the protein, from them one is chosen for further amplification to give rise to a titered Master virus stock, followed by preparation of working virus stocks. After initial small scale production is performed in Sf21 cells on rollers, a 10 L Biowave reactor is run at high cell density, using optimised conditions for production in Sf9 cells (3.3×106 cells/ml, 0.1 multiplicity of infection, additional yeast isolate feeding). After a 60 h infection period the cells are harvested by centrifugation and the aliquotted pellets are stored at −80° C.

Purification

Frozen Sf9 cell-pellets are resuspended in 8 volumes of ice-cold lysis buffer consisting of 50 mM Tris-HCl, pH 8.0, 500 mM NaCl, 10 mM b-mercaptoethanol, 0.6 mM PMSF and the protease inhibitor cocktail Complete a without EDTA (Roche Molecular Biochemicals). The suspension is homogenized by 10 strokes of Dounce homogenizer followed by three cycles of sonication of 1 min each. The cell lysate is centrifuged for 60 min at 15'000 G, filtered through a 3 mm Millipore filter and added to 20 ml Ni-NTA Superflow resin (Qiagen) pre-equilibrated in buffer A (50 mM Tris-HCl pH 8.0, 500 mM NaCl, 10 mM b-mercaptoethanol and 5 mM imidazole). After 2 h incubation at 4° C., the slurry is centrifuged, washed with buffer A and packed on a XK16/10 column. After baseline equilibration, proteins are eluted at 1 ml/min with a 70-min step gradient of buffer B (500 mM imidazole in A). The fractions are analysed by SDS-PAGE under reducing conditions, using 4-20% Tris-Glycine Novex gels. The fractions containing ROR are pooled, concentrated, and loaded on a size exclusion Superdex SPX7516/60 column (Pharmacia). The protein is separated at 1 ml/min in 50 mM Tris-HCl pH 7.5, 100 mM NaCl, 5 mM DTT. The fractions are analysed by SDS-PAGE. The 34-kDa band is identified as ROR by amino-terminal sequencing and mass spectrometry. The concentration of purified ROR-LBD is measured by reverse phase HPLC using a Vydac C4 column #214TP5415 (300 Å, 5 μm, 4.6 mm i.d.×150 mm). The separation is done with a linear gradient of 10-100% solvent B in solvent A over 25 minutes at 40° C. Solvent A is 0.1% anhydrous trifluoroacetic acid in acetonitrile/water (1:9 by volume) and solvent B is 0.1% anhydrous trifluoroacetic acid in acetonitrile/water (9:1 by volume). The flow rate is 1 ml/min and the protein is detected by absorbance at 220 nm.

Control proteins (His)6ERα-LBD301-553 and (His)6cytohesin-1 are cloned and expressed in E. coli. Purification is done by Ni-NTA and size exclusion chromatography as described above.

Protein Characterization

Amino acid sequences are determined on a Hewlett Packard G1000A N-terminal Protein Sequencing System. The system performs automated Edman chemistry on protein samples retained on miniature adsorptive biphasic columns. An optimized chemistry method (double couple 3.0) is used to enhance chemical efficiency and minimize lags. Analysis of PTH-amino acids is performed on an on-line Hewlett Packard HP1090 HPLC System equipped with a ternary pumping system and a narrowbore (2.1 mm×25 cm) PTH column. Mass spectrometry is carried out using a Q-Tof (Micromass, Manchester, UK) quadrupole time-of-flight hybrid tandem mass spectrometer equipped with a Micromass Z-type electrospray ionization source (ESI). Acquisition mass range is typically m/z 500-2500. Data are recorded and processed using Masslynx software. Calibration of the 500-2500 m/z scale is achieved by using the multiply-charged ion peaks from a mixture of horse heart myoglobin (MW 16951.5) and bovine trypsinogen (MW 23981.0).

Biacore Assay

The interactions between RORα-LBD and the nuclear co-activator GRIP1 are analyzed in real time by surface plasmon resonance using a Biacore 2000 system. The biotinylated 24-mer GRIP1 peptide is immobilized on streptavidin coated SA-chip at 25° C., at a final concentration of 5, 20 and 50 μM. The peptide (50 μl) is injected at a flow rate of 5 μl/min. In one flow cell, an unrelated biotinylated peptide is injected as a negative control. Binding of ROR-LBD on the coated chip is done at a flow rate of 20 μl/min. After sample injection (50 μl), the chip is washed with the same volume of HBS buffer (10 mM HEPES, 0.15 M NaCl, 3 mM EDTA, 0.005% Surfactant P20, pH 7.4), and then regenerated by injecting 50 μl of 2.0 M NaCl. The association and dissociation rate constants, and the equilibrium constant (Kd) are determined using the BIA evaluation software version 3.0. Expression and purification of (His)6RORα-LBD269-556 in the Baculovirus System Following the generation of a recombinant baculovirus, the RORa-LBD (residues 269-556) are readily expressed in Sf21 insect cells, as verified by Western blotting. Upon subsequent scale-up of plaque-purified, i.e. homogenous virus and optimisation of expression by kinetic experiments at low and high cell density, two initial 4.5 L batches (2×10 g wcp) are produced in rollers, resulting in a total of 5 mg of protein purified by Ni-NTA chromatography followed by gel filtration on a SPX75 column. The protein runs as a monomer on the size exclusion chromatography. N-terminal sequence analysis shows that ˜85% of the N-terminus of the protein is blocked, but the remaining free N-terminus is homogeneous, starting with the expected sequence GHHHHHHVVING. Mass spectrometry analysis shows a molecular mass of 34'412, corresponding to an excess of mass of +45 compared to the expected molecular mass (34'367), confirming the acetylation of the N-terminus. A second peak is observed by HPLC (˜24% of total ROR) corresponded to a mass of 34'422. The material shows stability at 4° C. for several weeks, but aggregates upon freezing and thawing. Subsequently, a larger amount of material can be produced in a single 10 L Biwave reactor, resulting in sufficient cell mass to cover the production of recombinant RORa-LBD for assay purposes. From a 30 g wcp aliquot (corresponding to 1.5 L culture volume), around 19 mg of (His)6RORα-LBD269-556 is purified by Ni-NTA chromatography, followed by SPX75 size exclusion chromatography. MS analysis shows that the material produced by Sf9 cells is comparable to that produced by Sf21 cells.

Biacore Binding Assay of (His)6RORα-LBD269-556 to GRIP1 Peptide

Since RORα is an orphan receptor, there is no ligand-binding assay available for functional study. Therefore, the purified (His)6RORα-LBD269-556 is tested for binding to the coactivator GRIP1 by a Biacore assay. A biotinylated 24-mer peptide containing the leucine charged domain LxxLL of the second NR-box of GRIP1 (NR-2) is immobilized on streptavidin coated SA-chip at concentrations ranging from 5 to 50 μM. The latter concentration results in a saturated surface with ˜100 resonance units (RU) of immobilized peptide. A control surface containing the unrelated peptide blotinyl-DEVD-1AL is used to measure unspecific binding. (His)6RORa-LBD shows specific binding to the biotinylated GRIP1 peptide compared to the controls. Binding of RORa-LBD to GRIP1 peptide is dose dependent. The apparent Kd measured was ˜241 nM. The binding is fully inhibited by the addition of the non-biotinylated 20-mer GRIP1 peptide. The calculated IC50 is ˜3 μM. Biacore binding assay to GRIP1, comparison with (His)6ERa-LBD301-553 The ligand-induced modulation of ER/coactivator interactions can be used as a positive control for the Biacore assay. Like RORα-LBD, (His)6ERa-LBD301-553 shows specific binding to the immobilized GRIP1 peptide. The apparent Kd is ˜450 nM. Preincubation of the ER-LBD with E2 (10 μM) results in a 6.5-fold increase of binding affinity (Kd ˜70 nM) compared to the basal level. In contrast, addition of the ERα antagonist BA31257 results in no significant increase of binding affinity compared to E2. The binding of the ER-LBD/E2 complex to the immobilized peptide is fully inhibited by the addition of free non-bioffnylated GRIP1 peptide.

The N-terminal 6xHis-tagged RORα-LBD can be cloned and expressed in the Baculovirus system. The construct consists of the human RORα C-terminal ligand-binding domain, residues 304-556 as defined by homology analysis with other LBDs. This N-terminal extension is initially designed as a spacer to avoid steric hindrance when binding (His)6RORα-LBD to a Ni-NTA metal-affinity surface, such as that of a Ni-NTA chip for Biacore studies, or NI-NTA-sepharose for ligand fishing experiments. Optimization of the expression conditions using the Biowave reactor results in a high expression titer (>10 mg/L cell culture). The LBD is purified to homogeneity by a combination of Ni-NTA and size exclusion chromatography. The purified monomeric protein (>95% as assessed on Coomassle stained SDS-gels) is then used for the coactivator binding assay on the Biacore. A biotinylated 24-mer peptide of the coactivator GRIP1, containing the LxxLL motif of the NR-2 box is immobilized on a streptavidin coated SA-chip. The RORα-LBD shows specific binding to the peptide with an apparent affinity of ˜240 nM, confirming previous pull-down experiments showing interactions between RORα and GRIP1. Since no ligand for RORα is yet available to investigate in vitro ligand-induced coactivation, ERα-LBD can be used as positive control to measure the extent of ligand-dependent modulation of receptor/coactivator interactions in the present Biacore assay. The apparent binding affinity of ERα-LBD to GRIP1 peptide increases around 6.5-fold when adding an agonist like estradiol, but remains almost unchanged by adding an antagonist such as BA31257. A comparable modulation of ERα-LBD/coactivator interactions upon ligand binding can be shown by Biacore and fluorescence resonance energy transfer experiments (Suen et al., J. Biol Chem. 1998, 273:27645-27653, Zhou et al. Mol. Endocrinol 1998, 10:1594-1604). However, in contrast to our study, these experiments are done with other ER-coactivators, namely SRC-3 and SRC-1, respectively. Moreover, the entire domain of SRC-3 containing the three NR-boxes was immobilized on the chip, leading to higher enhancement of binding affinities upon E2 binding (>10-fold increase) in Suen et al. The recent X-ray structures of apo-LBD, as well as agonist/LBD and antagonist/LBD complexes of a few nuclear receptors have given new insights to explain the shift of affinity. Indeed, the binding of an agonist induces a conformational change and a stabilization of the LBD resulting in a cognate surface for co-activator interaction, whereas pure antagonists with their bulky side chains prevent this change and thus coactivator interaction (Bourguet et al Trends Pharmacol Sci, 2000 21:381-388). Since this groove encompasses the highly conserved signature motif, most nuclear receptors have the potential to interact with coactivator NR-boxes, suggesting common mechanisms of ligand dependent nuclear receptor/coactivator interactions.

### Example 2

### Identification of Natural Ligands of Retinoic Acid Receptor Related Orphan Receptor (ROR)a.

(His)6RORa-LBD269-556 and (His)6RORa-LBD304-556 are produced in eukaryotic, insect Sf9 cells and purified according to procedures described in Example 1. Good quality crystals are only obtained from the extended (His)6RORa-LBD304-556 construct. Both constructs show identical biological activity. Construct (His)6RORa-LBD269-556 is chosen for MS experiments (ESI-MS and GO-MS). The protein is stored in Tris-HCl buffer at a concentration of 135 mM. Prior to mass spectrometry, buffer is exchanged by size exclusion chromatography (SEC) to a 50 mM ammonium acetate solution pH 7.0. SEC is performed with disposable spin-columns. CentrieSpin 20 columns (Princeton Separations, Adelphia, N.J.) are hydrated with 50 mM ammonium acetate buffer (pH 7.0) according to the manufacturer's instructions, and 60 *l of the protein solution (0.27 mg) are applied on the column. The column is spun at 750×g for 2 min, and the material collected is buffer exchanged on a second CentrieSpin column using the same procedure. The final concentration of the protein is determined by rHPLC and corresponded to approximately 50% of the initial concentration.

X-Ray Structure Elucidation

Crystals are obtained for the construct (His)6RORa-LBD304-556 and diffraction data are collected to 1.88 Å at the synchrotron (SNBL, ESRF Grenoble). The structure is solved ab initio with a Hg-derivative. After model building of the protein and water molecules, excellent electron density is still unaccounted for in the LBP which allowed the unambiguous identification of the ligand as cholesterol.

Ligand Exchange: Cholesterol by Cholesterol Sulfate

Cholesterol sulfate (MW 466.7, CholestSO4H, Sigma) is dissolved at 50 mM in DMSO and added at 2.5 mM final concentration to the (His)6RORa-LBD269-556 solution (135 μM). The resulting solution is incubated overnight at 4° C. and buffer is further exchanged according to the procedure described above. A control experiment incubating the same amount of RORa-LBD protein with 5% DMSO under identical conditions is performed.

Electrospray Ionization Mass Spectrometry

Mass spectrometry is carried out using a Q-Tof (Micromass, Manchester, UK) quadrupole time-of-flight hybrid tandem mass spectrometer equipped with a Micromass Z-type electrospray ionization source (ESI). The acquisition mass range is typically m/z 1500-4500 in 5 seconds. Calibration is achieved by using the multiply-charged ion peaks from hen egg lysozyme (Sigma; MW 14305.1 Da). The mass spectrometer is tuned in order to allow detection of multiply-charged species of non-covalent complexes. The source block temperature and desolvation temperature are kept at 50° C. and 80° C., respectively. Sample cone voltage (Vc) is set to 23 volts for standard measurements. In-source induced fragmentation experiments are performed by increasing Vc up to 100 volts. The protein solution is infused at a flow rate of 10 mL/min. Data are recorded and processed using Masslynx software. Spectra are deconvoluted using MaxEnt analysis software (Micromass, Manchester, UK)

Extraction Procedure and Derivatization

Extraction is performed by mixing 2 ml of a solution containing (His)6RORa-LBD269-556 at 4.6 mg/ml and 2 ml of hexane and shaking for 1 minute. The aqueous phase is extracted a second time using the same procedure. The organic phases are pooled and evaporated to dryness under nitrogen. The extract is dissolved in 50 ml pyridine. An aliquot of 10 ml is derivatized with 5 ml N,O-bistrimethylsilyl-trifluoroacetamide (BSTFA) at 60° C. for 30 min. Reference compounds cholesterol and 7-dehydrocholesterol (Sigma) are derivatized following the same procedure. For structure elucidation, the underivatized and the derivatized sample and reference compounds are analyzed by GC/MS, respectively.

Gas Chromatography—Mass Spectrometry (GC-MS)

The GC/MS system consists of a Carlo Erba Mega 5160 gas chromatograph, which is coupled to a Finnigan TSQ-700 mass spectrometer equipped with an electron impact ion source (EI). The ion source is heated to a temperature of 150° C., filament current is 20 mA, electron multiplier voltage 1000V, and the conversion dynode is set to 15 kV. The scan range is m/z 100-700 with a scan time of 2 seconds. The gas chromatographic separation is performed on a 10 m×0.2 mm Duran glass column coated with a 0.15 mm film of SDPE-08 using hydrogen as carrier gas at a constant flow rate of 2.5 ml/min. The temperature program is set to 100-330° C. at a rate of 6° C./min. The GC/MS interface temperature is set to 350° C.

High Resolution X-Ray Crystallography

The high-resolution (1.88 Å) of the X-ray data from the crystal of (His)6RORa-LBD304-556 shows the unexpected presence of a ligand in the LBP. The excellent fit to the electron density allows the identification of the ligand to be cholesterol. In addition, the X-ray structure shows all the 3D-details of the interactions in the LBP. Based on the X-ray structure, proposals are made for cholesterol derivatives, and in particular cholesterol sulfate is proposed as a ligand with a higher affinity to RORa than cholesterol, because of the electrostatic interactions with two Arg-side chains at the hydrophilic end of the LBP. Confirmation of the presence of cholesterol and comparative binding studies between cholesterol and cholesterol sulfate are further achieved by mass spectrometry on construct (His)6RORa-LBD269-556.

ESI-MS of (His)6RORa-LBD269-556

Previous reports describing the MS of non-covalent complexes (reviews: Pramanik et al 1998, J. Mass Spectrom. 33, 911-920, Veenstra et al 1999 Biophysical Chemistry, 79, 63-79) show that preservation of the native conformation of the protein is crucial for the detection of non-covalent complexes. Physiological conditions must be approximated and organic solvents must be avoided. Solvent conditions such as ionic strength, pH and counterions strongly influence the formation of gas phase ions (Lemaire et al 2001, Anal Chem, 73, 1699-1706). The protein concentration must be mmolar in order to avoid protein aggregation during the ionization process. Finally, electrospray ionization conditions such as source temperature, flow rate and official potential must be controlled to avoid collision-induced dissociation of the complex. The MaxEnt deconvoluted spectrum of (His)6RORa-LBD269-556 (15 mM in 50 mM ammonium acetate, pH 7.0) recorded at Vc=20 volts. The spectra yields two major molecular weights, e.g. 34411 Da and 34797 Da, respectively. MW 34411 Da (A) corresponds to the expected MW of (His)6RORa-LBD269-556. MW 34797 Da (B) corresponds to an additional adduct of 386 Da on the protein. This adduct disappears when cone voltage is increased to 35 volts indicating a weak binding between the protein and this adduct and a disruption of the bound protein-ligand under collision-induced dissociation in the atmosphere-vacuum interface. An average molecular weight of 386.3±0.5 Da is deduced from the difference between m/z values of multiply-charged ion species from compound A (MW 34411 Da) and compound B (34797Da), respectively. An MW search performed in the Chapman and Hall database of natural products yields 375 compounds with a mass between 385.8 and 386.8 Da. Restricting the search to steroids, as previously suggested by X-Ray analysis, scores 11 compounds. Among these compounds, cholesterol and cholesterol analogs are present. Further analyses by GC-MS are undertaken in order to more precisely identify the ligand and confirm the presence of a steroid.

GC-MS of Extract

The GC-MS analysis (SIC) of the extract obtained after derivatization with BSTFA shows two major steroids (R.T. 24.9 minutes and 25.7 minutes) and one minor steroid (R.T. 26.6 minutes). The +EI mass spectra of the underivatized and derivatized major compounds are almost identical to reference compounds cholesterol and 7-dehydrocholesterol (Provitamin D3). Additionally, cholesterol and 7-dehydrocholesterol are co-injected with the extract and show co-elution with the respective peaks. The +EI mass spectrum of the mono-trimethylsilylated minor steroid shows an abundant molecular ion at m/z 474.8. The losses of a methyl radical (m/z 459.0) and trimethylsilanol (m/z 384.4) are observed. The base peak at m/z 368.6 is formed by loss of a methyl radical with subsequent elimination of trimethylsilanol. Additionally, the loss of an ethyl radical is observed at m/z 445.7. The +EI mass spectrum of the underivatized minor steroid does not yield any molecular ion. The ion at m/z 384.3 is formed by loss of water. In addition, fragments of higher abundance are observed at m/z 369.7 (=m/z 384.3−methyl radical), m/z 366.7 (=m/z 384.3−H2O) and m/z 355.7 (=m/z 384.3−ethyl radical). Due to these observations an exact identification of this compound is not possible, but with a high probability this compound is a hydroxylated cholesterol. The position of the hydroxyl group cannot be located.

All in all, cholesterol and 7-dehydrocholesterol are identified in the extract of (His)6RORa-LBD269-556 by GC/MS. Additionally, a minor compound can be characterized as hydroxycholesterol. Proposed structures and SIC-% of the respective compounds are:

(i) Cholesterol: MW=386.7, SIC %=77.4

(ii) 7-Dehydrocholesterol (Provitamin D3):MW=384.7, SIC %=18.3

(iii) Hydroxycholesterol: MW=402.7, SIC %=4.2

1. A method of identifying a ligand to a nuclear receptor protein comprising:
(i) expressing a nuclear receptor protein in an eukaryotic expression system in the presence of at least one candidate ligand;
(ii) purifying and isolating the nuclear receptor protein;
(iii) measuring the spectra and molecular weight of the protein and protein-ligand complex by mass spectrometry to determine the presence of the ligand;
(iv) isolating the ligand and measuring the molecular weight of the ligand by mass spectrometry and comparing the measured mass spectra of the ligand with the mass spectra of compounds in known compound libraries to identify the ligand.

2. A method according to claim 1 wherein said nuclear receptor protein comprises a ligand binding domain.

3. A method according to claim 1 wherein said expression of step (i) comprises preferably a baculovirus expression system using insect cells.

4. A method according to claim 1 wherein step (i) occurs in the presence of at least one coregulator which is coexpressed.

5. A method according to claim 1 wherein step (ii) occurs in the presence of at least one coregulator.

6. A method according to claim 1 wherein both step (i) and step (ii) occur in the presence of at least one coregulator.

7. A method according to claim 1 wherein said mass spectrometry method of step (iii) for determining the presence of the ligand preferably comprises: continuous or pulsed electrospray ionization, or matrix assisted laser desorption ionization (MALDI).

8. A method according to claim 1 wherein said mass spectrometry method for identifying the ligand preferably comprises: continuous or pulsed electrospray ionization (ESI), atmospheric pressure chemical ionization (APCI), matrix assisted laser desorption ionization (MALDI), electron impact ionization (EI), or chemical ionization (Cl) mass spectrometry.

9. A method according to claim 1 step (iv) wherein said library comprises small molecule organic compounds.

10. A method according to claim 1 for use in a high throughput screen to identify ligands to nuclear receptor proteins.
